(19) United States
(12) Patent Application Publication
(10) Pub. No.: US 2025/0099291 A1
(43) Pub. Date: Mar. 27, 2025

(54) OSTOMY BAG UNDERGARMENT

(71) Applicant: Awestomy Active Corporation, Fort Lauderdale, FL (US)

(72) Inventor: Susan MOSEY, Juno Beach, FL (US)

(21) Appl. No.: 18/889,607

(22) Filed: Sep. 19, 2024

Related U.S. Application Data
(60) Provisional application No. 63/584,276, filed on Sep. 21, 2023.

(51) Int. Cl. A61F 5/449; A41D 13/12

(52) U.S. Cl. CPC A61F 5/449; A41D 13/1254

(57) ABSTRACT

A comfortable undergarment that holds ostomy bags in various positions without compressing the bag itself during use. The present invention uses performance materials and fabrics that provide breathability in a variety of flesh tones. The fabric is treated with Silver Fuze Technology for permanent anti-microbial protection. The ostomy bag support garment includes flatlock sticking to provide a low profile and smooth seams for no-chafe wear. The ostomy bag support garment comes in various sizes to fit each individual somatotype and in two types of profiles—low profile and full coverage. The ostomy bag support garment is machine washable.

### CROSS-REFERENCE TO RELATED APPLICATIONS

This application is related to and claims priority to U.S. Provisional Patent Application No. 62/841,0591, entitled “OSTOMY BAG UNDERGARMENT” filed on Sep. 21, 2023, both of which are assigned to the same assignee as this application and the teachings of which is hereby incorporated by reference in its entirety.

### FIELD OF THE INVENTION

This disclosure generally relates to a garment for supporting an ostomy bag and, more particularly, to an adjustable undergarment for holding ostomy bags in various positions.

### BACKGROUND

An ostomy is a surgery that creates an opening in the abdomen, changing the way that waste exits your body. This procedure is used to treat various diseases of the urinary or digestive systems. Individuals undergoing ostomy-type surgical operations have an outside opening or stoma in the skin of their body, which is then usually connected to an ostomy bag for receiving wastes from the body. This stoma is usually in the front lower torso area of the body. The stoma is usually a very delicate device and does not have sufficient strength to support an ostomy bag.

The stoma and ostomy bag on the individual often need to be accessed for inspection, maintenance, cleaning, and connection/disconnection of the ostomy bag from the stoma. The stoma is often very delicate and the applicant has found that moving an ostomy support garment to gain access to the stoma and/or ostomy bag can often be difficult and cause injury to the individual or user of the garment.

### SUMMARY

The present invention is a novel and comfortable undergarment that holds ostomy bags in various positions without compressing the bag itself during use. The present invention uses performance materials and fabrics that provide breathability in a variety of flesh tones. The fabric is treated with Silver Fuze Technology for permanent anti-microbial protection. The ostomy bag support garment includes flatlock sticking to provide a low profile and smooth seams for no-chafe wear.

The present invention comes in a variety of sizes to fit each individual somatotype and in two types of profiles—low profile and full coverage. The ostomy bag support garment is machine washable.

In one example, the undergarment includes a torso pocket portion shaped to surround a front torso area of a user. The torso pocket portion includes an inside material portion and an outside material portion, the torso pocket portion also includes a top edge, a bottom edge, a right side, and a left side, and the torso pocket portion includes a first end strap coupled to the right side and a second end strap coupled to the left side. The bottom edge may be longer in length than the top edge, thereby forming a contour on each of the right side and the left side to fit around the hips of the user. The right side includes seam stitching on the right side to join the inside material portion and an outside material portion. Similarly, the left side includes a left side seam stitching along the, joining together the inside material portion and an outside material portion. The right side seam stitching and the left side seam stitching may be a flatlock stitch. The bottom edge includes a bottom seam that joins together the inside material portion and an outside material portion between the right side and the left side. The bottom seam may be a shirring stitch to allow the outside material portion to extend outward away from the user.

The first end strap and the second end strap include complementary fasteners to join together around a back torso area of the user, the torso pocket portion including a top edge disposed near the top edge joining together the inside material portion and an outside material portion between the right side and the left side. For example, the complementary fasteners may be hook and loop fasteners that are disposed in a direction that is substantially perpendicular to the top edge.

The undergarment further includes an inner pocket formed in between the inside material portion and the outside material portion in the torso pocket portion around the front torso area of the user. The inside material portion, the outside material portion, or both may be treated with an anti-microbial treatment.

The undergarment further includes a rectangular opening formed along the inside material portion extending substantially parallel to a top edge to provide access to the inner pocket. The height of the rectangular opening is typically sized to fit a collar portion of an ostomy bag that couples with a stoma of the user. An elastic binding may be formed around at least a portion of the rectangular opening.

The undergarment may further include an ostomy bag with a collar. The position of the ostomy bag in the torso pocket portion is a stoma of the user on i) a right side with the position of the bag in a substantially vertical orientation; ii) a left side with the position of the bag in a substantially vertical orientation; iii) a right side with the position of the bag in a substantially horizontal orientation; and iv) a left side with the position of the bag in a substantially horizontal orientation.

The inside material portion, the outside material portion, or both are made from polyester sandex jersey in various grams and per square meter weights.

The inside material portion, the outside material portion, or both are made in a variety of flesh tone colors.

### BRIEF DESCRIPTION OF THE DRAWINGS

FIG. 1 is a front view of a user wearing a first example of an undergarment, according to an aspect of the invention;

FIG. 2 is a front view of the undergarment from FIG. 1 in the closed position, according to an aspect of the invention;

FIG. 3 is a back view of the undergarment from FIG. 1 in the closed position, according to an aspect of the invention;

FIG. 4 is a front view of the undergarment from FIG. 1 in the open position, according to an aspect of the invention;

FIG. 5 is an inside view of the undergarment from FIG. 1 in the open position with the bag placed in a first position, according to an aspect of the invention;

FIG. 6 is an inside view of the undergarment from FIG. 1 in the open position with the bag placed in a second position, according to an aspect of the invention;

FIG. 7 is an inside view of the undergarment from FIG. 1 in the open position with the bag placed in a third position, according to an aspect of the invention;

FIG. 8 is an inside view of the undergarment from FIG. 1 in the open position with the bag placed in a fourth position, according to an aspect of the invention;

FIG. 9 is a front view of a user wearing a second example of an undergarment, according to an aspect of the invention;

FIG. 10 is a front view of the undergarment from FIG. 9 in the closed position, according to an aspect of the invention;

FIG. 11 is a back view of the undergarment from FIG. 9 in the closed position, according to an aspect of the invention;

FIG. 12 is a front view of the undergarment from FIG. 9 in the open position, according to an aspect of the invention;

FIG. 13 is an inside view of the undergarment from FIG. 9 in the open position with the bag placed in a first position, according to an aspect of the invention;

FIG. 14 is an inside view of the undergarment from FIG. 9 in the open position with the bag placed in a second position, according to an aspect of the invention;

FIG. 15 is an inside view of the undergarment from FIG. 9 in the open position with the bag placed in a third position, according to an aspect of the invention; and

FIG. 16 is an inside view of the undergarment from FIG. 9 in the open position, with the bag placed in a fourth position, according to an aspect of the invention.

### DETAILED DESCRIPTION OF THE INVENTION

As required, detailed embodiments are disclosed herein; however, it is to be understood that the disclosed embodiments are merely examples and that the systems and methods described below can be embodied in various forms. Therefore, specific structural and functional details disclosed herein are not to be interpreted as limiting but merely as a basis for the claims and as a representative basis for teaching one skilled in the art to variously employ the disclosed subject matter in virtually any appropriately detailed structure and function. Further, the terms and phrases used herein are not intended to be limiting but rather to provide an understandable description.

The present invention is a novel and comfortable undergarment that holds ostomy bags in various positions without compressing the bag itself during use. The present invention uses performance materials and fabrics that provide breathability in a variety of flesh tones. The fabric is treated with Silver Fuze Technology for permanent anti-microbial protection. The ostomy bag support garment includes flatlock sticking to provide a low profile and smooth seams for no-chafe wear.

The present invention comes in a variety of sizes to fit each individual somatotype and in two types of profiles—low profile and full coverage. The ostomy bag support garment is machine washable.

### Non-Limiting Terminology

The terms “a” or “an,” as used herein, are defined as one or more than one. The term plurality, as used herein, is defined as two or more than two. The term another, as used herein, is defined as at least a second or more.

The term “and” in the phrase “one of A, B, and C” means either A or B or C or any combination of A, B, and C.

The term “anti-microbial protection” is a coating applied to materials, such as those available from Fuze Technologies based in Salt Lake City, Utah.

The term “coupled” or “coupling,” as used herein, is defined as “connected” although not necessarily directly and not necessarily mechanically. Coupled can be sewn using stitching of various forms, glue, welding, heat sealing, stapling, and other mechanical, physical or chemical means.

The term “flatlock” is a type of stitching that can be created in three-thread or two-thread forms and, as its name implies, results in a flat seam. A flatlock can be used in a variety of instances. The key advantage is that it has very little bulk because the raw edges of the material are completely encased in the stitching and looper threads.

The terms “including” and “having” as used herein are defined as comprising (i.e., open language).

The term “material” includes textiles made from various fibers as well as non-fiber-based materials.

The term “shirr” or “shirring” is a sewing technique in which an area of material or part of a garment is gathered together by means of drawn or elasticized threads.

The term “substantially” means to a large degree but not absolute.

Various materials or combinations of materials can be used to construct the mounting collar assembly and its components. For example, materials such as metals, alloys, composites, plastics, ceramics, and other inorganic or organic materials or combinations thereof may be used.

### First Example of Ostomy Undergarment Worn By A User

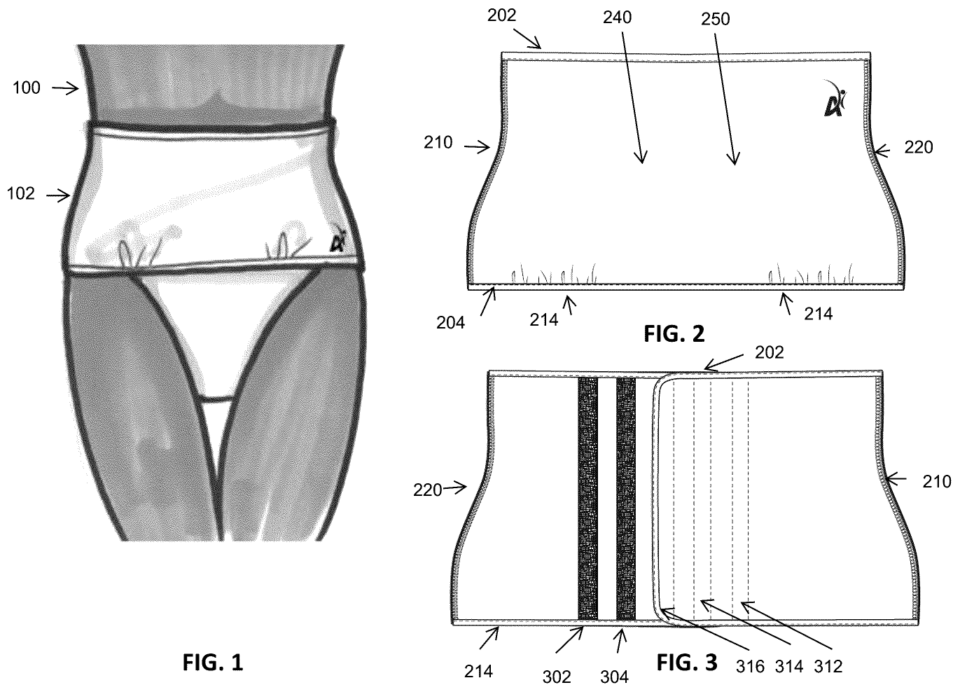

Referring to the drawings, and in particular to FIG. 1, is a front view of a user 100 wearing an undergarment 102 around her/his hips that holds a bag, such as an ostomy bag (not shown in this Figure).

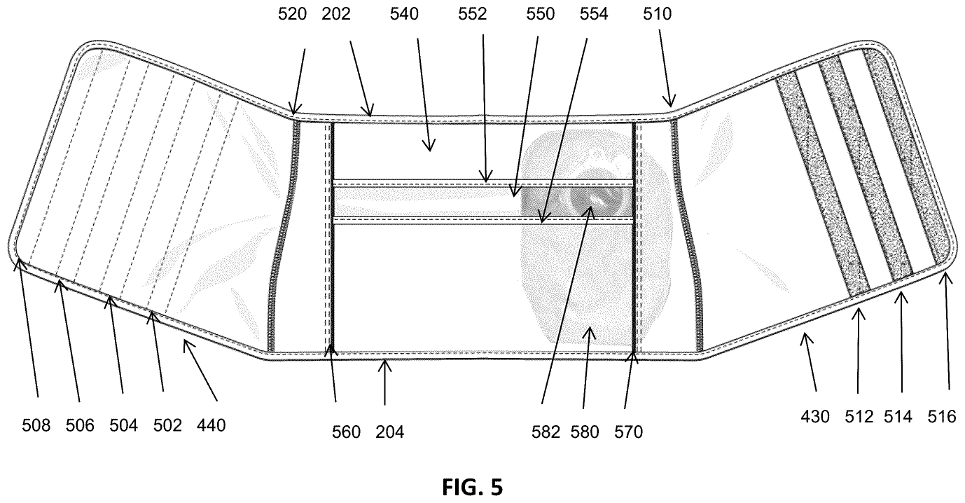

FIG. 2 is a front view of the undergarment 102 of FIG. 1 in a closed position typically positioned to surround a front torso area of the user 100. The undergarment 102 shown includes a torso pocket portion 250 created by coupling, such as sewing, an inside material portion (540 shown in FIG. 5), and an outside material portion 240 of material together, as will be explained and illustrated further below. This torso pocket portion 250 is an inner pocket formed between the inside material portion 540 and the outside material portion 240. Shown is a top edge 202 and a bottom edge 204. In this example, the bottom edge 204 may include elastic or a shirring stitch portion 214, as further described below.

The undergarment 102 shown also includes, from the user's perspective, a right side 210, a left side 220. In this example, the right side 210 and left side 220 are contour-shaped because the bottom edge 204 is longer in length, as shown, than the top edge 202 to better fit around the user's hips.

The material used to form the undergarment is a fabric. An example of the fabric is Polyester Spandex Jersey, which is in various gsm (grams per sqr meter) weights. In one example, the fabric may be made in a variety of flesh tones, like (stockings) undergarments or shapewear that are offered in flesh tones, in order to further blend in with the skin color of the user. In another example, the fabric may be treated with an anti-microbial treatment such as Silver Fuze Technology for permanent nano-particle technology protection. This treatment (coating) eliminates bacterial growth, has heat sink and, wicking properties, helping to keep the wearer cool, dry, and odor-free.

FIG. 3 is a back view of the undergarment from FIG. 1 in a closed position typically positioned to surround a back torso area of the user 102. A plurality of hook 302, 304 fasteners disposed of onto the outside fabric portion 240, i.e., the soft side for a hook fastener and the complementary loop fastener, are shown. The hook 302, 304 fasteners are positioned substantially perpendicular to the top edge 202 and bottom edge 204. One brand of hook and loop fasteners is Velco® brand. Also shown is the position of the stitching patterns for loop fasteners 312, 314, 316 coupled to the outside fabric portion 240. The complementary hook and loop fasteners adjust the undergarment's size around the torso of the user 102 to accommodate various body types.

### Front Open View of the First Example

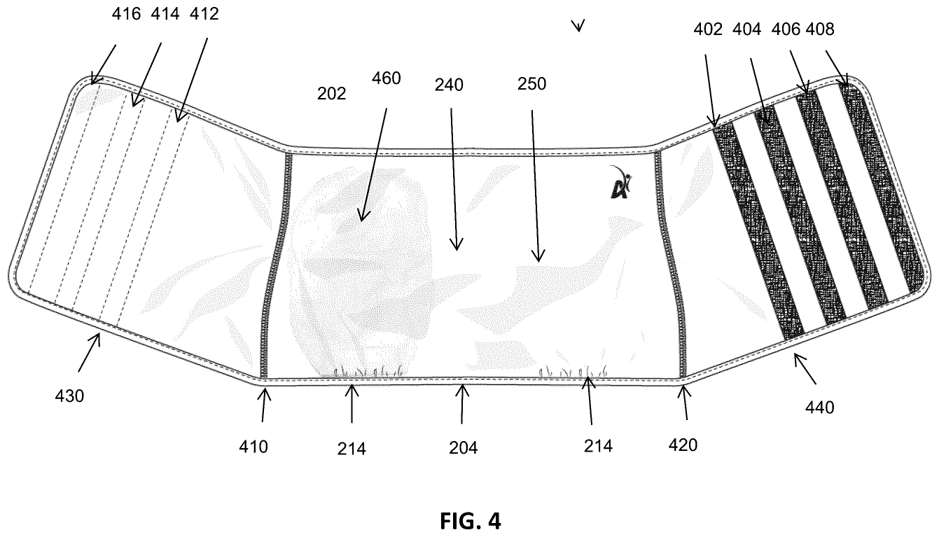

FIG. 4 is a front view of the undergarment from FIG. 1 in the open position. Shown is a left-side belt portion 440 coupled to the torso pocket portion 250. This is also shown on the right side portion 430 and a left side portion 440 coupled to the torso pocket portion 250. Also shown is the position of the bag in the torso pocket portion 250. This bag is shown in faint or light rendering because it is positioned internal to the torso pocket 250 and typically not visible through the outside fabric portion 240.

Continuing with FIG. 4, shown are a first hook piece 402 disposed on outside fabric portion 240, a second hook piece 404 disposed on outside fabric portion 240, and a third hook piece 406 disposed on outside fabric portion 240. The hook fasteners 402, 404, 406, 408 are positioned substantially perpendicular to the top edge 202 and bottom edge 204.

Also shown in FIG. 4 is a first set of stitching 412 for the first loop piece on the outside fabric portion 240. A second set of stitching 414 for a second loop piece on the outside fabric portion 240. A third set of stitching 416 for a second loop piece on the outside fabric portion 240.

A right-side seam 410 is disposed between the torso pocket portion 250 and the right end strap 430. Likewise, a left-side seam 420 is disposed between the torso pocket portion 250 and the left end strap 440. In one embodiment, scams 420 and 430 may made with flatlock stitching.

The seam along the bottom edge 204 may include a shirring stitch portion 214. The shirring portion allows the outside material portion 240 to extend outward away from the user 100 as the bag 460 begins to fill inside the torso pocket portion 250.

Back Open View of First Example with Various Positions of the Bag

FIG. 5 is an inside view of the undergarment from FIG. 1 in the open position, with the bag placed in a first position. Shown is stitching for the first loop piece 502 on inside material portion 540, stitching for the second loop piece 504 on inside material portion 540, stitching for the third loop piece 506 on inside material portion 540, and stitching for the fourth loop piece 508 on inside material portion 540.

Shown also is a first hook piece 512 on the inside material portion 540, a second hook piece 514 on the inside material portion, a third hook piece 516 on the inside material portion 540.

A left-side seam 510 is shown between the left end strap 440 and the torso pocket portion 250. A right-side seam 520 is shown between the right end strap 520 and torso pocket portion 250.

A rectangular opening 550 formed on the inside material portion 250 is substantially parallel to the top edge 202, as shown to provide access to that inside of the torso pocket portion 250, formed between the outside fabric portion 240 and the inside material portion 540. The rectangular opening has a top edge 552 and a bottom edge 554. The top edge 552 and bottom edge 554 can be fabricated with elastic bindings to allow the user to easily open the rectangular opening to place the bag 580 into the torso pocket portion 250. In addition, the use of elastic seams accommodates the collar 582 of the bag 580. The rectangular opening is also bounded by a left seam 560 and a right seam 570 as shown.

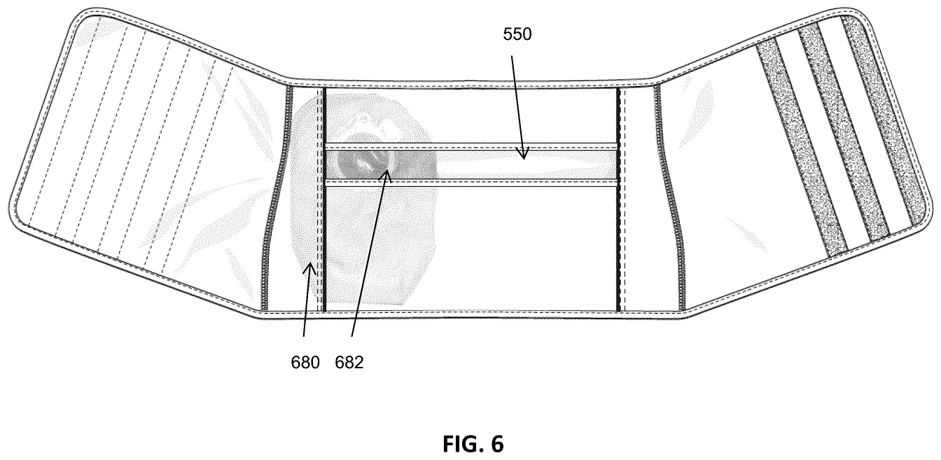

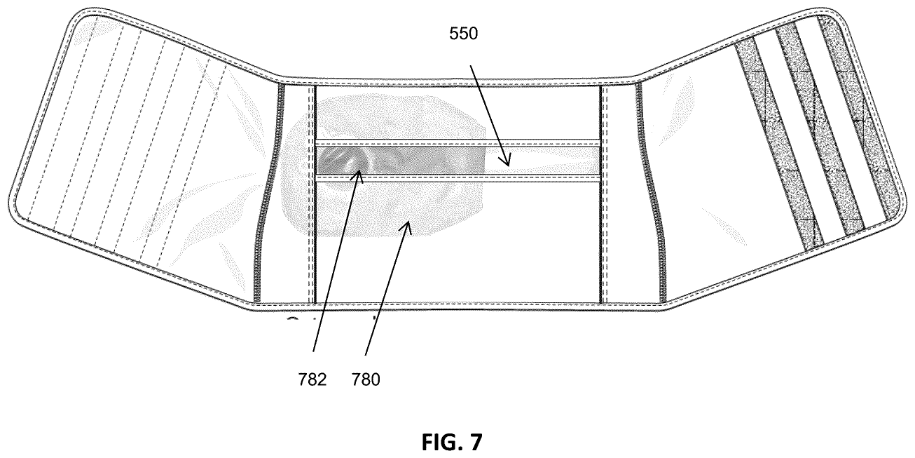

The bag 580 is shown in a first placement inside the torso pocket portion 250, with the collar 582 placed inside the rectangular opening 550. The bag 580 in this example is positioned on the right side of the user. The width of both the rectangular opening 550 and torso pocket allows the bag 580 and collar 582 to be configured in a variety of other positions as shown in FIG. 6 through FIG. 9.

FIG. 6 is an inside view of the undergarment from FIG. 1 in the open position, with a bag 680 and collar 682 placed in a second position. The bag 680 in this example is positioned on the left side of the user.

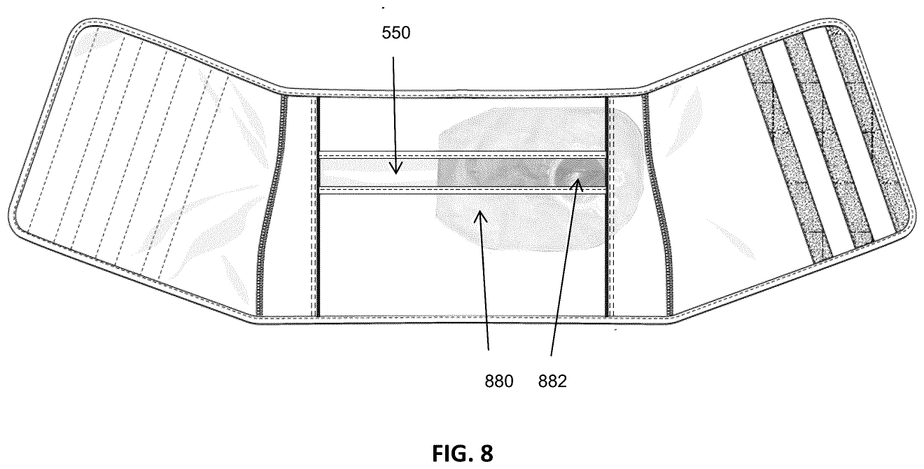

FIG. 7 is an inside view of the undergarment from FIG. 1 in the open position, with a bag 780 and collar 782 placed in a third position. The bag 780 in this example is positioned on the left side of the user.

FIG. 8 is an inside view of the undergarment from FIG. 1 in the open position with a bag 880 and collar 882 placed in a fourth position, according to an aspect of the invention.

### Approximate Non-Limiting Dimensions for the First Example of Undergarment

The following table provides an understanding of the approximate size of a medium-sized undergarment in this first example. It is important to note that the present invention is not limited to these sizes and is included solely to assist with understanding of the present invention.

| | | |
|---|---|---|
| 1 | Waist: Relaxed across Top Edge (Closed) | 14½″ |
| 2 | :Relaxed across 2″ down from Top Edge (Closed) | 13½″ |
| 3 | Hip Width Across along Bottom Edge (Closed) | 16½″ |
| 4 | Total Length: Side to Side (Open along Top) 202 | 31″ |
| 5 | Total Length: Side to Side (Open | 38″ |
| | along Bottom) 204 | |
| 6 | Back Overlap | 3¼″ |
| 7 | Total Height | 8½″ |
| 8 | Elastic Edge Binding | ¼″+ ¼″ |
| | (Satin fold Elastic to be sewn flat) | |
| 9 | Slight Shirring: 3″ from CF spread over 1½″ | |
| 10 | Logo Placement: Down from top edge | ¾″ |
| 11 | Over from Side Seam to edge of “A” | 1½″ |
| | Inside Construction: | |
| 12 | Top Lip Height 552 | 2½″ |
| 13 | Bottom Lip Height 554 | 4¾″ |
| 14 | Space between Lips 550 | 1¼″ |
| 15 | Lips width from side to side 550 | 12″ |
| 16 | Velcro Width | ¾″ |
| 17 | Sewn 3-4 rows ¾″ apart on back overlap panels | |

### Second Example of Ostomy Undergarment Worn By A User

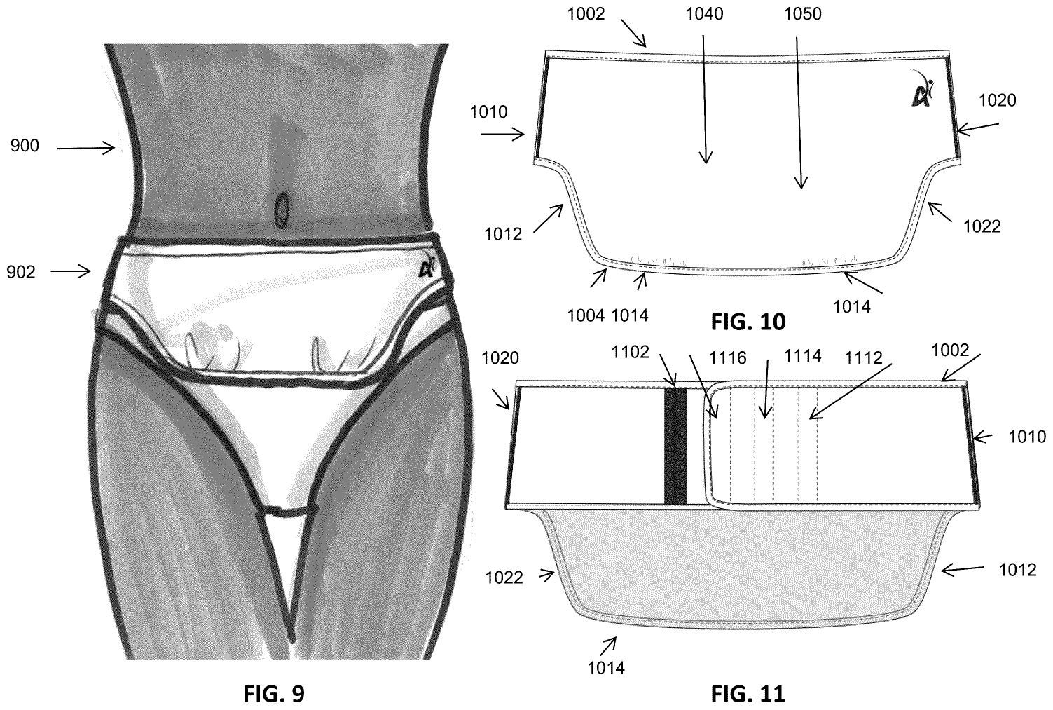

Items referred to in this embodiment for FIGS. 9-16 are similar to the items referenced above in FIG. 1-8. The last two digits of any reference number refer to a feature previously described above. For example, user 902 is similar to user 102, in which the last two digits are “02.”

Referring to the drawings, and in particular to FIG. 9, is a front view of a user 902 wearing an undergarment 900 around her/his hips that holds a bag, such as an ostomy bag (not shown in this Figure). As further described below, the major differences in this second example compared with the first example is the shape 1012 and 1022 of the bottom edge 1014 for the torso pocket portion 1050 and the height of the right side 1010 and left side 1020 compared to the height of the torso pocket portion 1050.

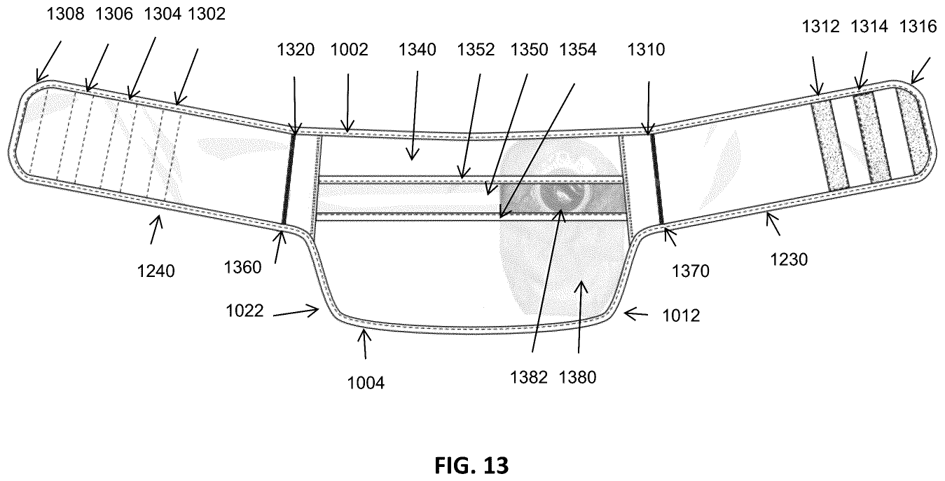

FIG. 10 is a front view of the undergarment 900 of FIG. 9 in a closed position typically positioned to surround a front torso area of the user 902. The undergarment 900 shown includes a torso pocket portion 250 created by couplings, such as sewing, an inside material portion (540 shown in FIG. 13), and an outside material portion 240 of material together, as will be explained and illustrated further below. This torso pocket portion 250 is an inner pocket formed between the inside material portion 1340 and the outside material portion 240. Shown is a top edge 202 and a bottom edge 204. In this example, the bottom edge 204 may include clastic or a shirring stitch portion 214, as further described below.

The undergarment 900 shown also includes, from the user's perspective, a right side 1010, a left side 1020. In this example, the right side 1010 and left side 1020 are contour-shaped because the bottom edge 1004 is longer in length, as shown, than the top edge 1002 to better fit around the user's hips.

The material used to form the undergarment is a fabric. An example of the fabric is Polyester Spandex Jersey in various gsm (grams per sqr meter) weights. In one example, the fabric may be made in a variety of flesh tones, like (stockings) undergarments or shapewear that are offered in flesh tones, in order to further blend in with the skin color of the user. In another example, the fabric may be treated with an anti-microbial treatment such as Silver Fuze Technology for permanent nano-particle technology protection. This treatment (coating) eliminates bacterial growth, has heat sink properties, and wicking properties, helping to keep the wearer cool, dry and odor-free.

FIG. 11 is a back view of the undergarment from FIG. 9 in a closed position typically positioned to surround a back torso area of the user 902. A plurality of hook 1102, 1104 fasteners disposed of onto the outside fabric portion 1040, i.e., the soft side for a hook fastener and the complementary loop fastener, are shown. The hook 1102, 1104 fasteners are positioned substantially perpendicular to the top edge 1002 and bottom edge 1004. One brand of hook and loop fasteners is Velco® brand. Also shown is the position of the stitching patterns for loop fasteners 1112, 1114, 1116 coupled to the outside fabric portion 1040. Again, the complementary hook and loop fasteners adjust the undergarment's size around the torso of the user 902 to accommodate various body types.

### Front Open View of the Second Example

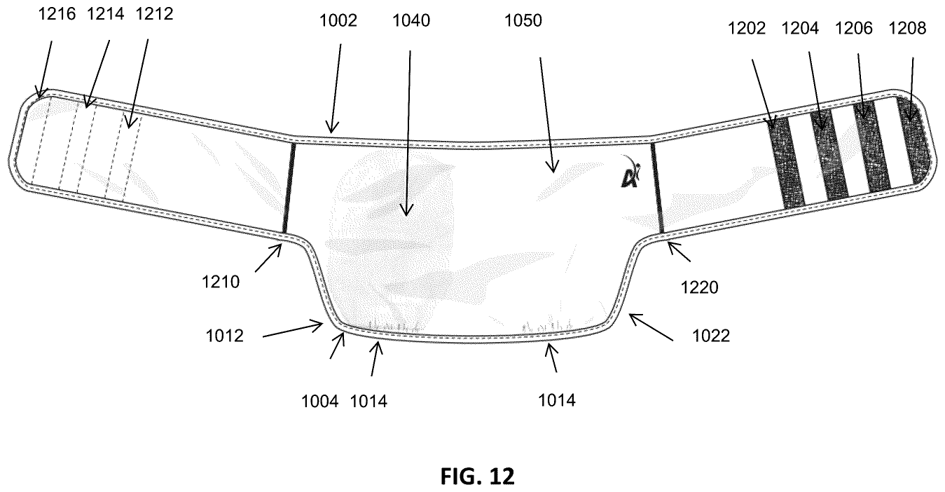

FIG. 12 is a front view of the undergarment from FIG. 9 in the open position. Shown is a left-side belt portion 1230 coupled to the torso pocket portion 1050. This is also shown on the right side portion 1230 and a left side portion 1240 coupled to the torso pocket portion 1050. Also shown is the position of the bag in the torso pocket portion 1050. This bag is shown in faint or light rendering because it is positioned internal to the torso pocket 1050 and typically not visible through the outside fabric portion 1040.

Continuing with FIG. 12, shown are a first hook 1202 piece disposed on outside fabric portion 1040, a second hook piece 1204 disposed on outside fabric portion 1040, and a third hook piece 1206 on disposed outside fabric portion 1040. The hook fasteners 1202, 1204, 1206, 1208 are positioned substantially perpendicular to the top edge 1002 and bottom edge 1004.

Also shown in FIG. 12 is a first set of stitching 1212 for first loop piece on the outside fabric portion 1040. A second set of stitching 1214 for a second loop piece on outside fabric portion 1040. A third set of stitching 1216 for a second loop piece on outside fabric portion 1040.

Note that shapes 1012 and 1022 of the bottom edge 1014 for the torso pocket portion 1050. Also, note the height of the right side 1010 and left side 1020 compared to the height of the torso pocket portion 1050. This provides a lower profile design as compared with the first example.

A right-side seam 1210 is disposed between the torso pocket portion 1040 and the right end strap 1230. Likewise, a left-side seam 1220 is disposed between the torso pocket portion 1040 and the left end strap 1240. In one embodiment, scams 1220 and 1230 may made with flatlock stitching.

The seam along the bottom edge 1004 may include a shirring stitch portion 1014. The shirring portion allows the outside material portion 1040 to extend outward away from the user 900 as the bag begins to fill inside the torso pocket portion 1050.

Back Open View of the Second Example with Various Positions of the Bag

FIG. 13 is an inside view of the undergarment from FIG. 9 in the open position, with the bag placed in a first position. Shown is stitching for the first loop piece 1302 on inside material portion 1340, stitching for the second loop piece 1304 on inside material portion 1340, stitching for the third loop piece 1306 on inside material portion 1340, and stitching for the fourth loop piece 1308 on inside material portion 1340.

Shown also is a first hook piece 1312 on the inside material portion 1340, a second hook piece 1314 on the inside material portion, a third hook piece 1316 on the inside material portion 1340.

A left-side seam 1310 is shown between the left end strap 1240 and the torso pocket portion 1050. A right-side seam 1320 is shown between the right end strap 1320 and torso pocket portion 1050.

A rectangular opening 1350 formed on the inside material portion 1050 is substantially parallel to the top edge 1002, as shown to provide access to that inside of the torso pocket portion 1050, formed between the outside fabric portion 1040 and the inside material portion 1340. The rectangular opening has a top edge 1352 and a bottom edge 1354. The top edge 1352 and bottom edge 1354 can be fabricated with elastic bindings to allow the user to easily open the rectangular opening to place the bag 1380 into the torso pocket portion 1050. In addition, the use of elastic seams accommodates the collar 1382 of the bag 1380. The rectangular opening is also bounded by a left seam 1360 and a right seam 1370 as shown.

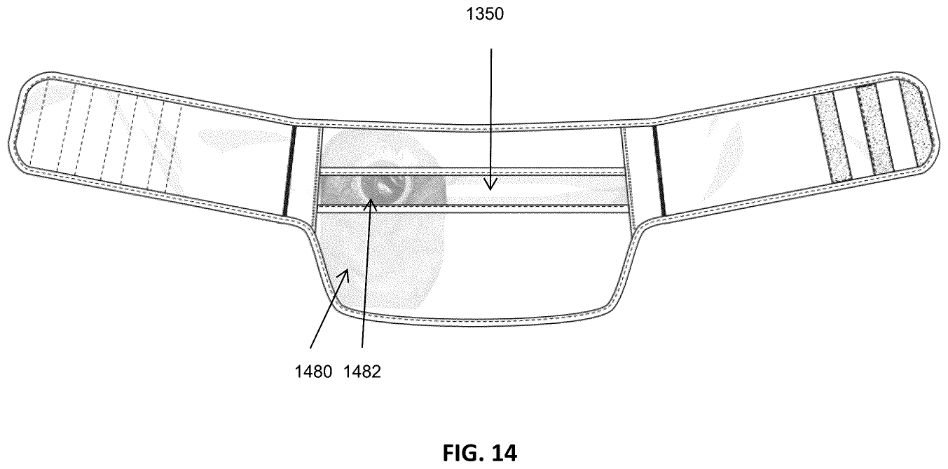

The bag 1380 is shown in a first placement inside the torso pocket portion 1050, with the collar 1382 placed inside the rectangular opening 1350. The bag 1380 in this example is positioned on the right side of the user. The width of both the rectangular opening 1350 and torso pocket allows the bag 1380 and collar 1382 to be configured in a variety of other positions as shown in FIG. 14 through FIG. 9.

FIG. 14 is an inside view of the undergarment from FIG. 9 in the open position, with a bag 1480 and collar 1482 placed in a second position. The bag 1480 in this example is positioned on the left side of the user.

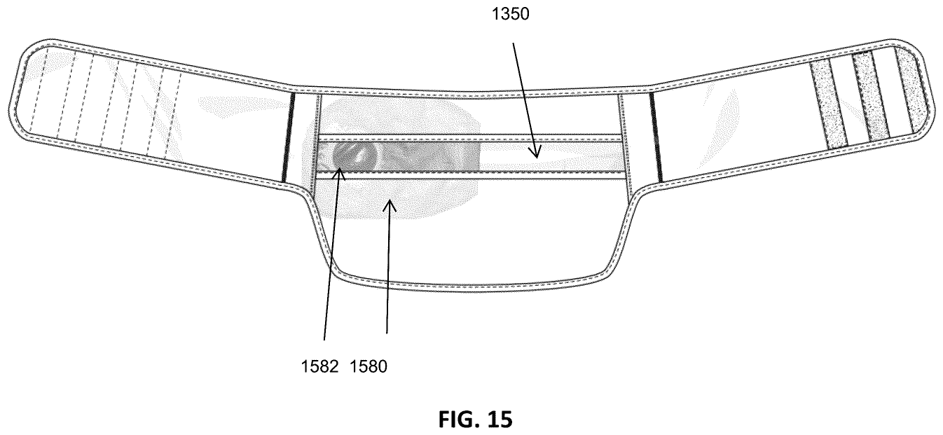

FIG. 15 is an inside view of the undergarment from FIG. 9 in the open position, with a bag 1580 and collar 1582 placed in a third position. The bag 1580 in this example is positioned on the left side of the user.

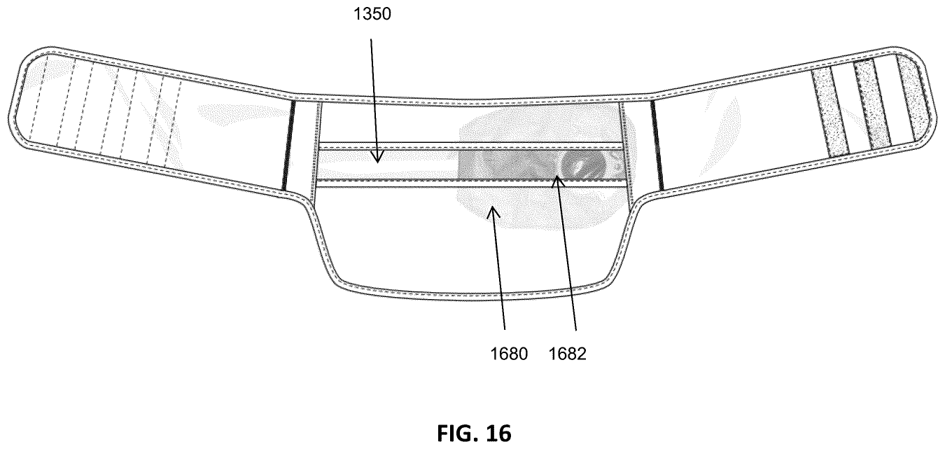

FIG. 16 is an inside view of the undergarment from FIG. 9 in the open position with a bag 1680 and collar 1682 placed in a fourth position, according to an aspect of the invention.

### Approximate Non-Limiting Dimensions for the Second Example of Undergarment

The following table provides an understanding of the approximate size of a medium-sized undergarment in this first example. It is important to note that the present invention is not limited to these sizes and is included solely to assist with understanding of the present invention.

### Point of Measure (POM)

| POM | iMeas Description | M |
|---|---|---|
| 1 | Waist: Relaxed across Top Edge (Closed) | 13¾″ |
| 2 | Total Length: Side to Side (Open along Top) | 33″ |
| 3 | Back Overlap | 4½″ |
| 4 | Total Height @ Center Front (CF) | 7½″ |
| 5 | Total Height 6″ over from CF | 3½″ |
| | (Wrap Strap Height) | |
| 6 | Elastic Edge Binding | ¼″ + ¼″ |
| | (Satin fold Elastic to be sewn flat) | |
| 7 | Slight Shirring across bottom edge | |
| 8 | Width across Bottom Edge before | 9½″ |
| | start of curve up to Strap | |
| 9 | Logo Placement: Down from Top | ¾″ |
| | Edge to top of the “A” | |
| 10 | From Side Seam Fold flat to edge of “A” | 1¼″ |
| | Inside Construction: | |
| 11 | Top Lip Height | 2″ |
| 12 | Bottom Lip Height | 4¼″ |
| 13 | Space between Lips | 1¼″ |
| 14 | Lips width from side to side | 12″ |
| 15 | Velcro Width | ¾″ |
| 16 | Sewn 3-4 rows ¾″ apart on back overlap panels | |

### Non-Limiting Examples

The present invention has been shown and described herein in what is considered to be the most practical and preferred embodiment. It is recognized, however, that departures may be made therefrom within the scope of the invention and that obvious modifications will occur to a person skilled in the art.

The illustrations of embodiments described herein are intended to provide a general understanding of the structure of various embodiments, and they are not intended to serve as a complete description of all the elements and features of apparatus and systems that might make use of the structures described herein. Many other embodiments will be apparent to those of skill in the art upon reviewing the above description. Other embodiments may be utilized and derived therefrom, such that structural and logical substitutions and changes may be made without departing from the scope of this disclosure. Figures are also merely representational and may not be drawn to scale. Certain proportions thereof may be exaggerated, while others may be minimized. Accordingly, the specification and drawings are to be regarded in an illustrative rather than a restrictive sense. Thus, although specific embodiments have been illustrated and described herein, it should be appreciated that any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description. Therefore, it is intended that the disclosure not be limited to the particular embodiment(s) disclosed as the best mode contemplated for carrying out this invention, but that the invention will include all embodiments falling within the scope of the appended claims.

What is claimed is:

1. An undergarment comprising:
a torso pocket portion shaped to surround a front torso area of a user, the torso pocket portion includes an inside material portion and an outside material portion, the torso pocket portion also includes a top edge, a bottom edge, a right side, and a left side, and the torso pocket portion includes a first end strap coupled to the right side and a second end strap coupled to the left side, the first end strap and the second end strap include complementary fasteners to join together around a back torso area of the user, the torso pocket portion including a top edge disposed near the top edge joining together the inside material portion and an outside material portion between the right side and the left side;
an inner pocket formed in between the inside material portion and the outside material portion in the torso pocket portion around the front torso area of the user; and
a rectangular opening formed along the inside material portion extending substantially parallel to a top edge to provide access to the inner pocket.

2. The undergarment of claim 1, wherein the complementary fasteners are hook and loop fasteners that are disposed in a direction that is substantially perpendicular to the top edge.

3. The undergarment of claim 1, wherein the inside material portion, the outside material portion, or both are treated with an anti-microbial treatment.

4. The undergarment of claim 1, wherein the bottom edge is longer in length than the top edge, thereby forming a contour on each of the right side and the left side to fit around the hips of the user.

5. The undergarment of claim 1, wherein a height of the rectangular opening is sized to fit a collar portion of an ostomy bag that couples with a stoma of the user.

6. The undergarment of claim 1, further comprising:
an elastic binding formed around at least a portion of the rectangular opening.

7. The undergarment of claim 1, further comprising:
a right side seam stitching along the right side, joining together the inside material portion and an outside material portion; and
a left side seam along the left side, joining together the inside material portion and an outside material portion.

8. The undergarment of claim 7, wherein the right side seam stitching and the left side seam stitching is a flatlock stitch.

9. The undergarment of claim 1, further comprising:
a bottom seam forming the bottom edge, joins together the inside material portion and an outside material portion between the right side and the left side.

10. The undergarment of claim 9, wherein the bottom seam is a shirring stitch to allow the outside material portion to extend outward away from the user.

11. The undergarment of claim 1, further comprising:
an ostomy bag with a collar;
a position of the ostomy bag in the torso pocket portion is a stoma of the user on
a right side with the position of the ostomy bag in a substantially vertical orientation;
a left side with the position of the ostomy bag in a substantially vertical orientation;
a right side with the position of the ostomy bag in a substantially horizontal orientation; and
a left side with the position of the ostomy bag in a substantially horizontal orientation.

12. The undergarment of claim 1, wherein the inside material portion, the outside material portion or both, are made from Polyester Spandex Jersey in various grams per square meter weights.

13. The undergarment of claim 12, wherein the inside material portion, the outside material portion or both are made in a variety of flesh tone colors.

14. The undergarment of claim 1, wherein a distance between a top edge and a bottom edge of the first end strap and the second end strap is less than a distance between the top edge and the bottom edge of the torso pocket portion.

15. An undergarment comprising:
a torso pocket portion shaped to surround a front torso area of a user, the torso pocket portion includes an inside material portion and an outside material portion, the torso pocket portion also includes a top edge, a bottom edge, a right side, and a left side, and the torso pocket portion includes a first end strap coupled to the right side and a second end strap coupled to the left side, the first end strap and the second end strap include complementary fasteners to join together around a back torso area of the user, the torso pocket portion including a top edge disposed near the top edge joining together the inside material portion and an outside material portion between the right side and the left side, and the top edge of the torso pocket portion is at substantially a same height as a top edge of the first end strap and as a top edge of the second end strap and a distance between the top edge and a bottom edge of the first end strap and the second end strap is less than a distance between the top edge and the bottom edge of the torso pocket portion;
an inner pocket formed in between the inside material portion and the outside material portion in the torso pocket portion around the front torso area of the user; and
a rectangular opening formed along the inside material portion extending substantially parallel to a top edge to provide access to the inner pocket.

16. The undergarment of claim 15, wherein the complementary fasteners are hook and loop fasteners that are disposed in a direction that is substantially perpendicular to the top edge.

17. The undergarment of claim 15, wherein the inside material portion, the outside material portion, or both are treated with an anti-microbial treatment.

18. The undergarment of claim 15 wherein the bottom edge is longer in length than the top edge, thereby forming a contour on each of the right side and the left side to fit around the hips of the user.

19. The undergarment of claim 15 wherein a height of the rectangular opening is sized to fit a collar portion of an ostomy bag that couples with a stoma of the user.

20. The undergarment of claim 15, further comprising:
an elastic binding formed around at least a portion of the rectangular opening.
